(19) United States
(12) Patent Application Publication
(10) Pub. No.: US 2021/0393924 A1
(43) Pub. Date: Dec. 23, 2021

(54) VASCULAR ACCESS INSTRUMENT AND RELATED DEVICES AND METHODS

(71) Applicant: Becton, Dickinson and Company, Franklin Lakes, NJ (US)

(72) Inventors: Jonathan Karl Burkholz, Salt Lake City, UT (US); Yiping Ma, Layton, UT (US); Weston F. Harding, Lehi, UT (US)

(21) Appl. No.: 17/330,218

(22) Filed: May 25, 2021

Related U.S. Application Data
(60) Provisional application No. 63/041,517, filed on Jun. 19, 2020.

(51) Int. Cl. A61M 25/09; A61M 25/06; A61M 39/02

(52) U.S. Cl. CPC A61M 25/09; A61M 25/0662; A61M 2025/0681; A61M 2025/09083; A61M 2039/0258; A61M 39/0247

(57) ABSTRACT

A vascular access system may include a catheter assembly, which may include a catheter adapter and a catheter extending distally from the catheter adapter. The vascular access system may include an instrument advancement device coupled to the catheter assembly. The instrument advancement device may include a vascular access instrument. The vascular access instrument may include a coil formed by a flat wire wound around an axis into multiple loops. The instrument advancement device may be configured to advance the vascular access instrument from a retracted position to an advanced position beyond a distal end of the catheter. The distal end of the catheter may include a distal opening. The coil may extend through the distal opening of the catheter in response to the vascular access instrument being in the advanced position.

### RELATED APPLICATIONS

This application claims benefit of U.S. Provisional Patent Application No. 63/041,517, filed on Jun. 19, 2020, entitled VASCULAR ACCESS INSTRUMENT AND RELATED DEVICES AND METHODS, which is incorporated herein in its entirety.

### BACKGROUND

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and the introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using the catheter may be difficult for several reasons, particularly when a dwell time of the catheter within the vasculature is more than one day. When the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, the catheter is often used for acquiring a blood sample at a time of catheter placement, but the catheter is less frequently used for acquiring a blood sample during the catheter dwell period. Therefore, when a blood sample is required, an additional needle stick is often used to provide vein access for blood collection, which may be painful for the patient and result in higher material costs.

In some instances, in order to avoid the additional needle stick, a vascular access instrument may be used to access the vasculature of the patient via the catheter. The vascular access instrument may be inserted through the catheter and into the vasculature to extend a life of the catheter and allow blood withdrawal through the catheter without the additional needle stick.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices. More particularly, the present disclosure relates to a vascular access instrument, which may be delivered through a catheter assembly to vasculature of a patient via an instrument advancement device. In some embodiments, the vascular access instrument may facilitate an increased dwell period of a catheter of the catheter assembly within the vasculature of the patient. In some embodiments, the instrument advancement device may be used to advance the vascular access instrument into the catheter and/or beyond a distal tip of the catheter when the catheter is compromised to overcome obstructions such as thrombus, valves, and/or a fibrin sheath in or around the catheter that may otherwise prevent blood draw. In some embodiments, the instrument advancement device may provide needle-free delivery of the vascular access instrument to the vasculature of the patient for blood collection, fluid delivery, patient or device monitoring, or other clinical needs by utilizing an existing catheter dwelling within the vasculature.

In some embodiments, the vascular access instrument may be configured to insert through a vascular access device, such as, for example, the catheter assembly. In some embodiments, the vascular access instrument may include a coil formed by a flat wire wound around an axis into multiple loops. As referred to in the present disclosure, the term “flat wire” may correspond to a wire that includes a first side and a second side opposite the first side, and the first side and/or the second side is planar prior to the wire being wound around the axis into loops during manufacture. In some embodiments, the first side may form an outer surface of the coil. In some embodiments, the second side may form an inner surface of the coil. In some embodiments, the core wire may be coupled to the inner surface of the coil. In some embodiments, each of the loops of the coil may be spaced apart from a next adjacent loop of the loops.

In some embodiments, the vascular access instrument may include a core wire extending through at least a portion of the coil. In some embodiments, the core wire may be cylindrical. In some embodiments, the core wire may be flat. In some embodiments, the core wire may be aligned with the axis. In some embodiments, the core wire may be offset from the axis.

In some embodiments, the vascular access instrument may include an elongated strip extending through the coil. In some embodiments, each of the loops may include a distal end and a proximal end. In some embodiments, the distal end of each of the loops and the proximal end of each of the loops contact the elongated strip. In some embodiments, the elongated strip may be linear and/or aligned with the axis.

In some embodiments, the vascular access instrument may include multiple support beams extending between the coil. In some embodiments, a first set of the support beams may be disposed along a first line parallel to the axis. In some embodiments, a second set of the support beams may be disposed along a second line parallel to the axis. In some embodiments, the first line may be spaced apart from the second line. In some embodiments, the first line may be 180 degrees apart from the second line.

In some embodiments, a third set of the support beams may be disposed along a third line parallel to the axis. In some embodiments, the first line, the second line, and the third line may be evenly spaced around a circumference of the coil. In some embodiments, a complete turn of the coil is disposed in between one or more of the following: next adjacent support beams of the first set, next adjacent support beams of the second set, and next adjacent support beams of the third set.

In some embodiments, the coil may include multiple other loops. In some embodiments, the other loop may be proximate the loops. In some embodiments, each of the other loops of the coil may contact a next adjacent loop of the other loops around a circumference of the next adjacent loop of the other loops.

In some embodiments, a vascular access system may include a catheter assembly, which may include a catheter adapter and a catheter extending distally from the catheter adapter. In some embodiments, an instrument advancement device may be coupled to the catheter assembly. In some embodiments, the instrument advancement device may include the vascular access instrument. In some embodiments, the instrument advancement device may be configured to advance the vascular access instrument from a retracted position to an advanced position beyond a distal end of the catheter. In some embodiments, the distal end of the catheter may include a distal opening. In some embodiments, the coil may extend through the distal opening of the catheter in response to the vascular access instrument being in the advanced position. In some embodiments, the distal end of the catheter may include one or more diffuser holes. In some embodiments, a gap in the core wire may be disposed at the distal opening of the catheter.

In some embodiments, another vascular access instrument configured to insert through the vascular access device may include a distal end, which may include a tubular element. In some embodiments, the tubular element may include one or more holes. In some embodiments, the other vascular access instrument may include a wire. In some embodiments, the other vascular access instrument may include a connector disposed between the tubular element and the wire. In some embodiments, the connector may include one or more other holes. In some embodiments, a distal end of the tubular element may be closed.

It is to be understood that both the foregoing general description and the following detailed description are examples and explanatory and are not restrictive of the present disclosure, as claimed. It should be understood that the various embodiments are not limited to the arrangements and vascular access instrumentality shown in the drawings. Also, the drawings are not necessarily to scale. It should also be understood that the embodiments may be combined. For example, one or more features of a particular vascular access instrument may be combined with one or more features of another particular vascular access instrument. It should also be understood that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example vascular access system, illustrating an example vascular access instrument in an example retracted position, according to some embodiments;

FIG. 1B is an upper perspective view of the vascular access system, illustrating the vascular access instrument in an example advanced position, according to some embodiments;

FIG. 1C is an enlarged upper perspective view of a portion of the vascular access system, illustrating the vascular access instrument in the advanced position, according to some embodiments;

FIG. 1D is an enlarged cutaway view of another portion of the vascular access system, illustrating the vascular access instrument in the advanced position, according to some embodiments;

FIG. 1E is a cross-sectional view of the vascular access system, illustrating the vascular access system in the advanced position within example vasculature, according to some embodiments;

FIG. 1F is an enlarged cross-sectional view of a portion of an example coil of the vascular access instrument, according to some embodiments;

FIG. 1G is an enlarged cutaway view of the vascular access instrument disposed within the vascular access system, illustrating the vascular instrument in the advanced position, according to some embodiments;

FIG. 1H is an enlarged cutaway view of the vascular access instrument disposed within the vascular access system, illustrating the vascular instrument in the advanced position, according to some embodiments;

FIG. 2 is a bar graph illustrating example sum of maximum shear ratio and example sum of blood collection rate ratio, according to some embodiments;

FIG. 3A is an upper perspective view of an example distal end of the vascular access instrument, according to some embodiments;

FIG. 3B is a cross-sectional view of the distal end of the vascular access instrument of FIG. 3A, according to some embodiments;

FIG. 3C is a transverse cross-sectional view of a portion of the vascular access instrument of FIG. 3A disposed within an example catheter, according to some embodiments;

FIG. 4A is an upper perspective view of another example distal end of the vascular access instrument, according to some embodiments;

FIG. 4B is a cross-sectional view of the portion of the vascular access instrument of FIG. 4A, according to some embodiments;

FIG. 5A is an upper perspective view of the vascular access instrument, illustrating an example elongated strip, according to some embodiments;

FIG. 5B is a top view of the vascular access instrument of FIG. 5A, according to some embodiments;

FIG. 6A is an upper perspective view of another example distal end of the vascular access instrument disposed within the vascular access system, illustrating the vascular instrument in the advanced position, according to some embodiments;

FIG. 6B is a cross-sectional view of the distal end of the vascular access instrument of FIG. 6A disposed within the vascular access system, illustrating the vascular instrument in the advanced position, according to some embodiments;

FIG. 6C is a cross-sectional view of a portion of the vascular access instrument of FIG. 6A, according to some embodiments;

FIG. 6D is an upper perspective view of the portion of the vascular access instrument of FIG. 6A, according to some embodiments;

FIG. 6E is a cross-sectional view of the vascular access instrument of FIG. 6A, according to some embodiments;

FIG. 7A is an upper perspective view of the vascular access instrument, according to some embodiments;

FIG. 7B is a cross-sectional view of the distal end of the vascular access instrument of FIG. 7A disposed within the vascular access system, illustrating the vascular instrument in the advanced position, according to some embodiments;

FIG. 7C is an enlarged upper perspective view of the distal end of the vascular access instrument of FIG. 7A disposed within the vascular access system, illustrating the vascular instrument in the advanced position, according to some embodiments;

FIG. 7D is an enlarged cross-sectional view of the distal end of the vascular access instrument of FIG. 7A disposed within the vascular access system, illustrating the vascular instrument in the advanced position, according to some embodiments;

FIG. 8 is a bar graph illustrating example average of maximum shear ratio and an example average blood collection rate ratio, according to some embodiments;

FIG. 9A is an upper perspective view of the vascular access instrument, according to some embodiments;

FIG. 9B is a cross-sectional view of the vascular access instrument of FIG. 9A extending from the catheter, according to some embodiments;

FIG. 9C is an enlarged cross-sectional view of a portion of the vascular access instrument of FIG. 9A, according to some embodiments;

FIG. 9D is an enlarged cross-sectional view of the vascular access instrument of FIG. 9A disposed within the vascular access system, illustrating the vascular instrument in the advanced position, according to some embodiments;

FIG. 10A is an upper perspective view of the vascular access instrument, according to some embodiments;

FIG. 10B is an upper perspective view of the vascular access instrument, according to some embodiments;

FIG. 10C is an upper perspective view of the vascular access instrument, according to some embodiments;

FIG. 10D is an upper perspective view of the vascular access instrument, according to some embodiments;

FIG. 10E is an upper perspective view of the vascular access instrument of FIG. 10D extending from the catheter in the advanced position, according to some embodiments;

FIG. 10F is an enlarged upper perspective view of the vascular access instrument of FIG. 10D extending from the catheter in the advanced position, according to some embodiments;

FIG. 11 is a cross-sectional view of another example vascular access instrument disposed in the advanced position within the catheter, according to some embodiments;

FIG. 12A is an upper perspective view of an upper perspective view of an example instrument advancement device, according to some embodiments; and

FIG. 12B is a cross-sectional view of the instrument advancement device, according to some embodiments.

### DESCRIPTION OF EMBODIMENTS

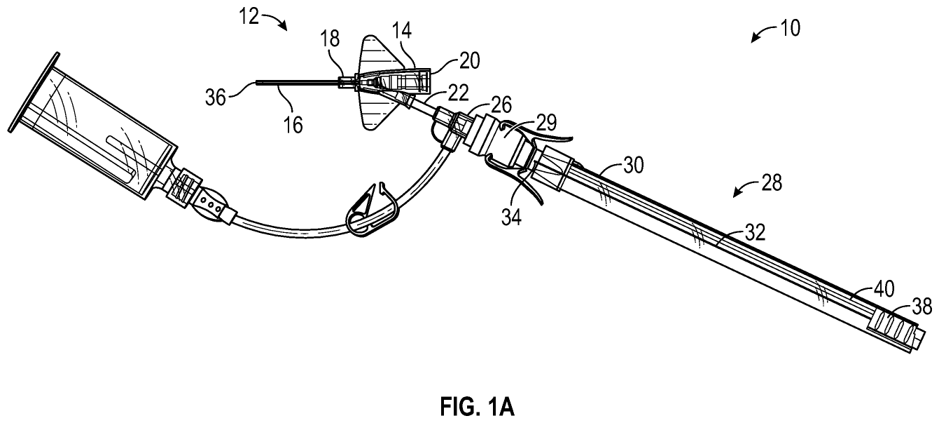

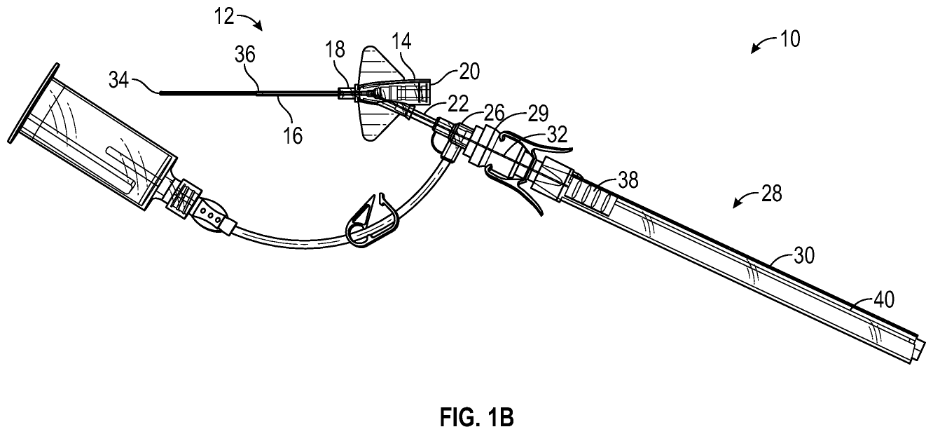

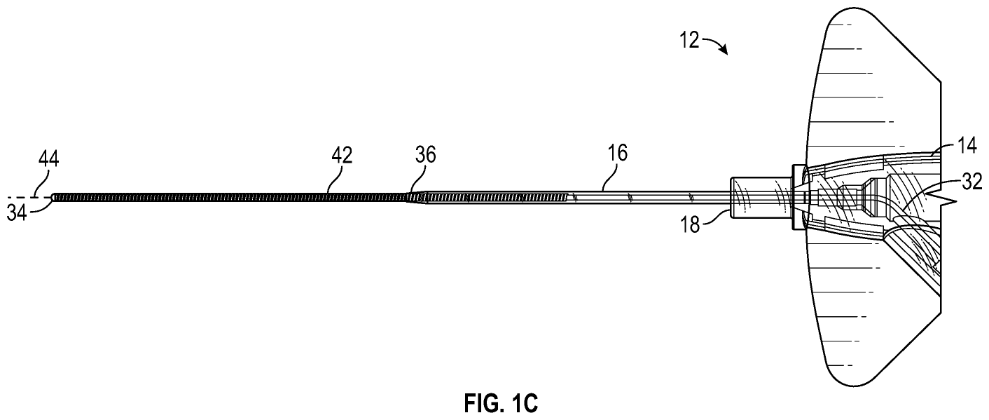

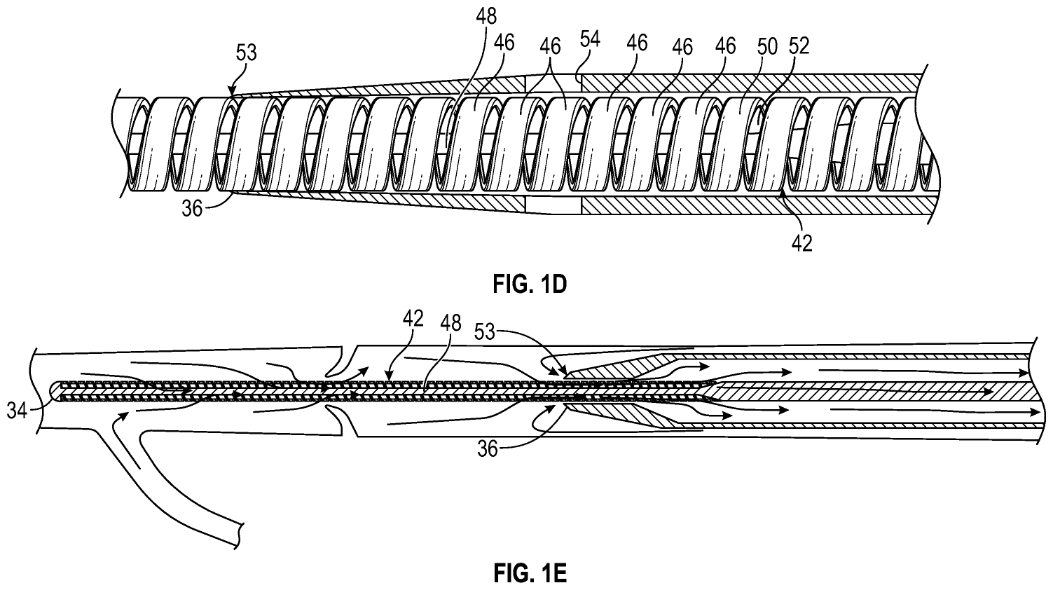

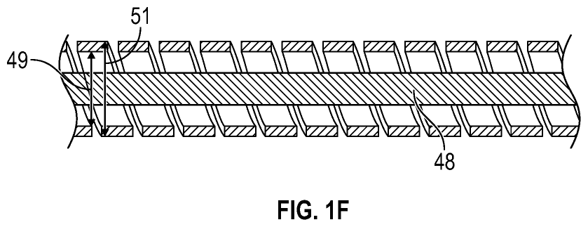

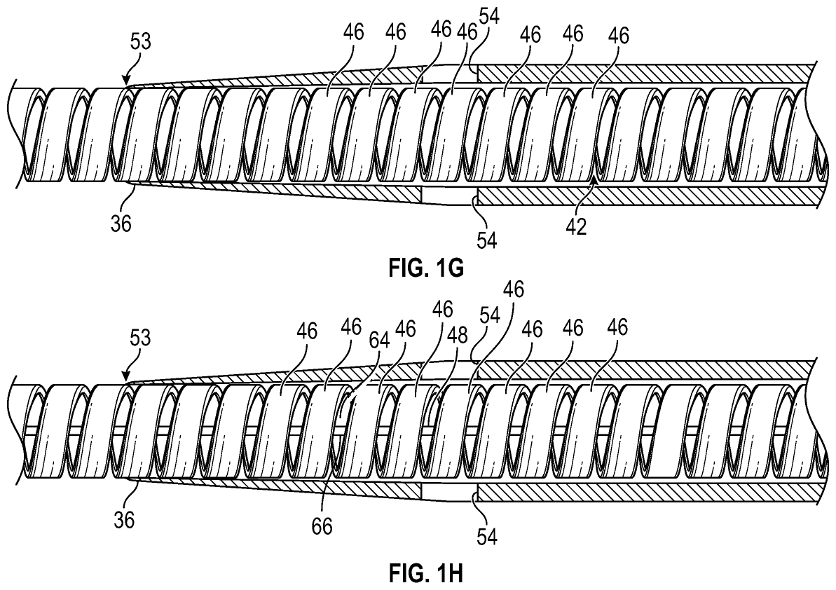

Referring now to FIGS. 1A-1B, a vascular access system 10 is illustrated, according to some embodiments. In some embodiments, the vascular access system 10 may include a catheter assembly 12, which may include a catheter adapter 14 and a catheter 16. In some embodiments, the catheter 16 may include a peripheral intravenous catheter, a peripherally-inserted central catheter, or a midline catheter. In some embodiments, the catheter adapter 14 may include a distal end 18, a proximal end 20, and a lumen extending through the distal end 18 and the proximal end 20. In some embodiments, the catheter 16 may extend distally from the distal end 18 of the catheter adapter 14.

In some embodiments, the catheter adapter 14 may be integrated with an extension tube 22, which may extend from a side port 24 of the catheter adapter 14. In some embodiments, an adapter 26, such as a Y-adapter or a T-adapter, for example, may be coupled to a proximal end of the extension tube 22.

In some embodiments, an instrument advancement device 28 may be coupled to the catheter assembly 12 in various ways. As an example, the instrument advancement device 28 may be coupled to a port of the adapter 26. As another example, the instrument advancement device 28 may be coupled to a needleless connector 29 disposed between the port of the adapter 26 and the instrument advancement device 28. As another example, the instrument advancement device 28 may be coupled to the proximal end 20 of the catheter adapter 14. In some embodiments, another extension tube and/or a blood collection device adapter may be coupled to another port of the adapter 26. In some embodiments, the blood collection device adapter may receive a blood collection device, such as, for example, a syringe or a blood collection tube.

In some embodiments, the instrument advancement device 28 may include a housing 30 configured to couple to the catheter assembly 12. In some embodiments, the instrument advancement device 28 may include a vascular access instrument 32. In some embodiments, the instrument advancement device 28 may include any suitable delivery device. Some examples of instrument advancement devices that may be used with the vascular access instrument 32 are described further in in U.S. patent application Ser. No. 16/037,246, filed Jul. 17, 2018, entitled “EXTENSION HOUSING A PROBE OR INTRAVENOUS CATHETER,” U.S. patent application Ser. No. 16/388,650, filed Apr. 18, 2019, entitled “INSTRUMENT DELIVERY DEVICE HAVING A ROTARY ELEMENT,” U.S. patent application Ser. No. 16/037,319, filed Jul. 17, 2018, entitled “MULTI-DIAMETER CATHETER AND RELATED DEVICES AND METHODS,” U.S. patent application Ser. No. 16/502,541, filed Jul. 3, 2019, entitled “DELIVERY DEVICE FOR A VASCULAR ACCESS INSTRUMENT,” U.S. patent application Ser. No. 16/691,217, filed Nov. 21, 2019, entitled “SYRINGE-BASED DELIVERY DEVICE FOR A VASCULAR ACCESS INSTRUMENT,” U.S. patent application Ser. No. 16/742,013, filed Jan. 14, 2020, entitled “CATHETER DELIVERY DEVICE AND RELATED SYSTEMS AND METHODS,” and U.S. patent application Ser. No. 16/838,831, filed Apr. 2, 2020, entitled “VASCULAR ACCESS INSTRUMENT HAVING A FLUID PERMEABLE STRUCTURE AND RELATED DEVICES AND METHODS,” which are each incorporated by reference in their entirety.

In some embodiments, the instrument advancement device 28 may be configured to introduce the vascular access instrument 32 into the catheter assembly 12. In some embodiments, in response to the vascular access instrument 32 being introduced into the catheter assembly 12, the vascular access instrument 32 may access a fluid path of the catheter assembly 12 and/or the vascular access instrument 32 may extend through the catheter assembly 12 to access the vasculature of the patient.

In some embodiments, the instrument advancement device 28 may be configured to advance the vascular access instrument 32 between a retracted position, illustrated, for example, in FIG. 1A, to an advanced position, illustrated, for example, in FIG. 1B. In some embodiments, a distal tip 34 of the vascular access instrument 32 may be disposed distal to a distal end 36 of the catheter 16 in response to the vascular access instrument 32 being in the advanced position. In some embodiments, in response to the vascular access instrument 32 being in the retracted position, the distal tip 34 of the vascular access instrument 32 may be disposed within the housing 30. In some embodiments, a proximal end of the vascular access instrument 32 may be coupled to an advancement tab 38, which may be gripped and moved along a slot 40 by a user to move the vascular access instrument 32 between the retracted position and the advanced position. The advancement tab 38 may extend through the slot 40, and a portion of the advancement tab 38 coupled to the proximal end of the vascular access instrument 32 may be within the housing 30.

In some embodiments, the catheter 16 may be constructed of fluorinated ethylene propylene, TEFLON™, silicon, thermoplastic elastomer, thermoplastic polyurethane, a fluorinated polymer, a hydrophilic material, a hydrophobic material, an anti-fouling material, or another suitable material. In some embodiments, the catheter 16 may include an anti-thrombogenic coating. In some embodiments, all or a portion of the vascular access instrument 32 may be constructed of metal or another suitable material. In some embodiments, the coil 42 may be constructed of metal or another suitable material. In some embodiments, the distal end 36 of the catheter 16 may be symmetric or asymmetric.

Referring now to FIGS. 1C-1F, in some embodiments, the vascular access instrument 32 may include a coil 42 formed by a flat wire wound around an axis 44 into multiple loops 46. In some embodiments, the coil 42 may be disposed at a distal end of the vascular access instrument 32 and/or adjacent the distal tip 34. In some embodiments, each of the loops 46 of the coil 42 may be spaced apart from a next adjacent loop of the loops 46, which may facilitate fluid permeability of the distal end of the vascular access instrument 32. In some embodiments, the coil 42 may provide multiple fluid pathways along a length of the vascular access instrument 32, which may facilitate entry of blood into the catheter assembly 12 from a portion of the vasculature more distant from the catheter 16. In some embodiments, the coil 42 and the fluid pathways along the length of the vascular access instrument 32 may facilitate increased flow rates of fluid through the vascular access instrument 32 and the catheter 16. In some embodiments, the coil 42 and the fluid pathways along the length of the vascular access instrument 32 may facilitate a reduced blood collection time. In some embodiments, the coil 42 may reduce a shear stress and related risk of hemolysis of blood moving into and/or through the catheter 16.

In some embodiments, the coil 42 may facilitate soft and gentle contact with a wall of the vasculature in response to insertion of the vascular access instrument 32 into the vasculature. In some embodiments, the coil 42 may reduce shear stress on fluid moving through the vascular access instrument 32.

In some embodiments, the vascular access instrument 32 may include a core wire 48 extending through at least a portion of the coil 42. In some embodiments, the core wire 48 may be cylindrical, as illustrated in FIGS. 1C-1F. In some embodiments, the core wire 48 may be aligned with the axis 44. In some embodiments, the core wire 48 may provide structural support to the vascular access instrument 32.

In some embodiments, the flat wire may include a first side 50 and a second side 52 opposite and parallel to the first side 50 prior to the flat wire being wound around the axis 44 into the loops 46 during manufacture. In some embodiments, the first side 50 may form an outer surface of the coil 42. In some embodiments, the second side 52 may form an inner surface of the coil 42. In some embodiments, the core wire 48 may be coupled to the inner surface of the coil 42.

In some embodiments, the flat wire may increase an inner diameter 49 of the coil 42 to facilitate an increase fluid flow rate through the vascular access instrument 32. In some embodiments, the flat wire may increase the inner diameter 49 of the coil 42 and still allow an outer diameter 51 of the coil 42 to be a same length as an outer diameter of a standard vascular access instrument.

In some embodiments, the distal end 36 of the catheter 16 may include a distal opening 53. In some embodiments, the coil 42 may extend through the distal opening 53 of the catheter 16 in response to the vascular access instrument 32 being in the advanced position. In some embodiments, the outer diameter 51 of the coil 42 may be less than a diameter of the distal opening 53, which may create a gap through which fluid may flow. In some embodiments, the distal end 36 of the catheter 16 may include one or more diffuser holes 54, which may be aligned with a portion of the vascular access instrument 32 that includes the coil 42 to facilitate blood flow into the catheter assembly 12 and/or fluid infusion into the vasculature.

In some embodiments, dimensions of the coil 42 may vary based on a gauge-size of the catheter 16, a stiffness of the vascular access instrument 32, a spacing between each of the loops 46 of the coil 42, a number or size of fluid pathways along the length of the vascular access instrument 32, or another factor.

Referring now to FIG. 1G, in some embodiments, the vascular access instrument 32 may not include the core wire 48, which may increase flow through the coil 42 and/or the catheter 16.

Referring now to FIG. 1H, in some embodiments, the core wire 48 may be flat, which may increase flow through the coil 42 and/or the catheter 16. In some embodiments, the flat wire may include a first side 64 and a second side 66 opposite the first side 64. In some embodiments, the first side 64 may be parallel to the second side 66.

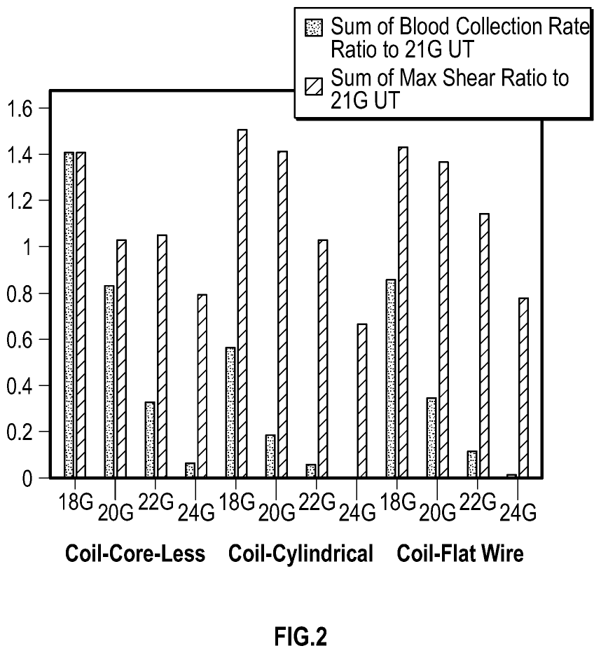

Referring now to FIG. 2, the bar graph illustrates, according to some embodiments, a sum of blood collection rate ratio to a 21G UT (“BD VACUTAINER™ ULTRATOUCH™”) and a sum of max shear ratio to a 21G UT (“BD VACUTAINER™ ULTRATOUCH™”) in a particular vascular access system with the coil 42 that includes the flat wire wound around the axis 44 to form the loops 46, another particular vascular access system with the coil 42 that includes a cylindrical wire wound around the axis 44 to form the loops 46, and another particular vascular access system without the core wire 48.

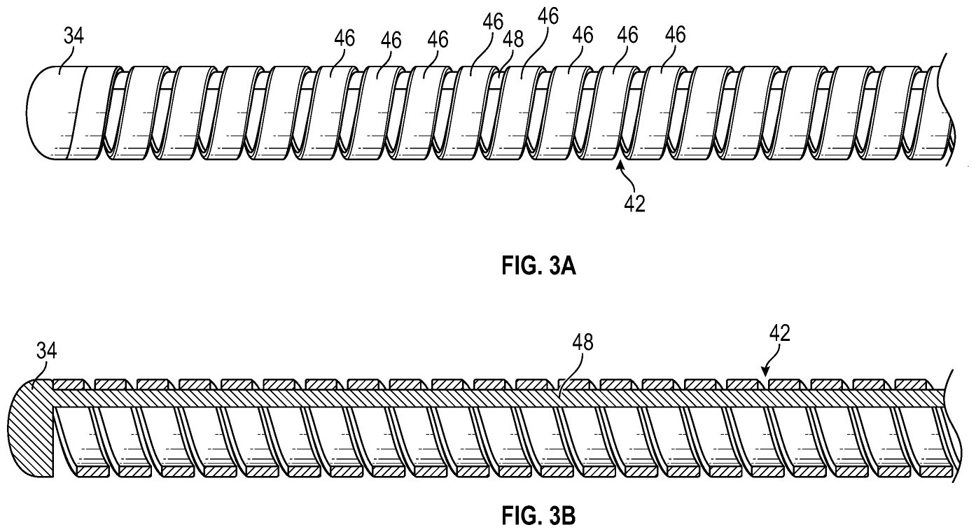

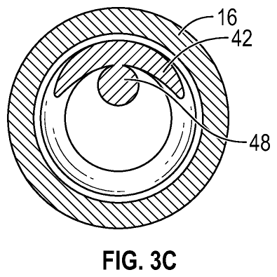

Referring now to FIGS. 3A-3C, in some embodiments, the core wire 48 may be offset from the axis 44, which may facilitate blood flow into the catheter assembly 12 and/or fluid infusion into the vasculature along the axis 44 and a central portion of the coil 42 and/or a central portion of the catheter 16. In some embodiments, the core wire 48 that is offset from the axis 44 may be coupled to the coil 42 at one or more points. In some embodiments, the core wire 48 may be coupled to the inner surface of the coil 42 at one or more points. For example, the core wire 48 may be bonded to the coil 42 or the inner surface of the coil 42. In some embodiments, the core wire 48 may be coated with a thin layer of a polymer material, heated while in contact with the coil 42, and allowed to cool to bond the core wire 48 to the coil 42 or the inner surface of the coil 42.

In some embodiments, the distal tip 34 may be rounded or blunt, which may prevent injury to the vasculature. In some embodiments, the distal tip 34 may be disposed at a distal end of the core wire 48 and/or monolithically formed with the distal end of the core wire 48 as a single unit. Additionally or alternatively, in some embodiments, the distal tip 34 may be coupled to a distal end of the coil 42.

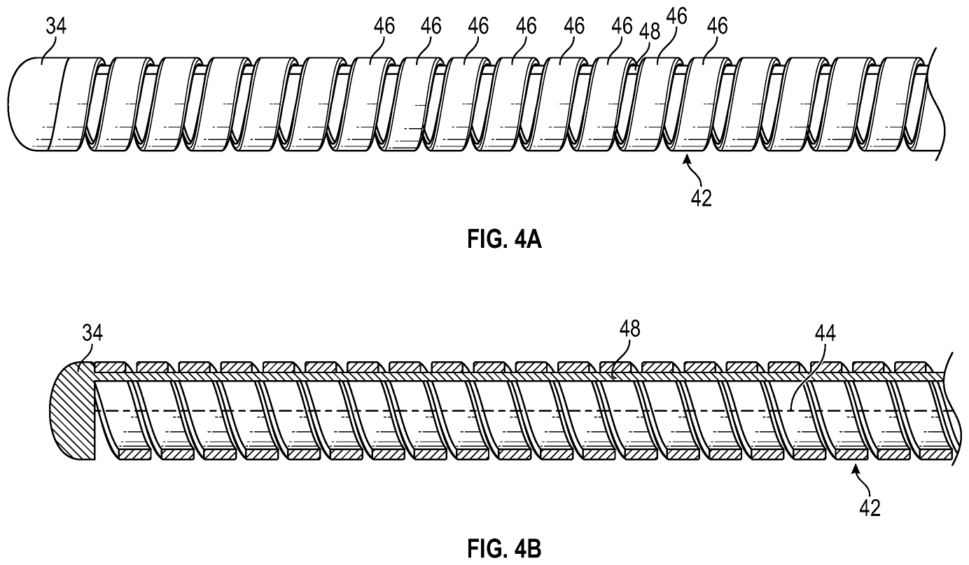

Referring now to FIGS. 4A-4B, in some embodiments, the core wire 48 may be offset from the axis 44 and/or flat, which may facilitate blood flow into the catheter assembly 12 and/or fluid infusion into the vasculature along the axis 44 and a central portion of the coil 42 and/or a central portion of the catheter 16. In some embodiments, the core wire 48 that is offset from the axis 44 may be coupled to the inner surface of the coil at one or more points.

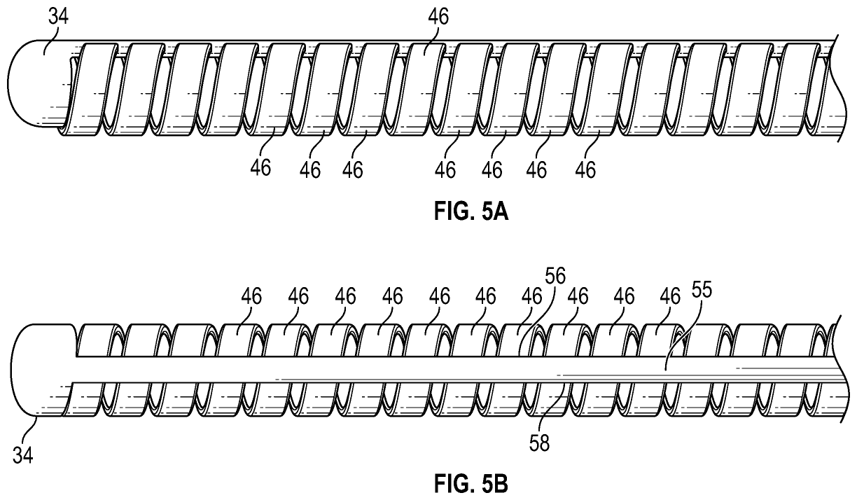

Referring now to FIGS. 5A-5B, in some embodiments, the vascular access instrument 32 may include an elongated strip 55 extending through the coil 42. In some embodiments, each of the loops 46 may include a distal end 56 and a proximal end 58. In some embodiments, the distal end 56 of each of the loops 46 and the proximal end 58 of each of the loops 46 contact the elongated strip 55. In some embodiments, the elongated strip 55 may be linear and/or aligned with the axis 44. In some embodiments, the elongated strip 55 may provide structural support to the vascular access instrument 32. In some embodiments, the elongated strip 55 may extend along all or a portion of the coil 42. In some embodiments, the elongated strip 55 may be disposed on any of the top, bottom, or side of the vascular access instrument 32.

In some embodiments, the distal tip 34 may be rounded or blunt. In some embodiments, the distal tip 34 may be disposed at a distal end of the elongated strip 55 and/or monolithically formed with the distal end of the elongated strip 55 as a single unit. Additionally or alternatively, in some embodiments, the distal tip 34 may be coupled to a distal end of the coil 42.

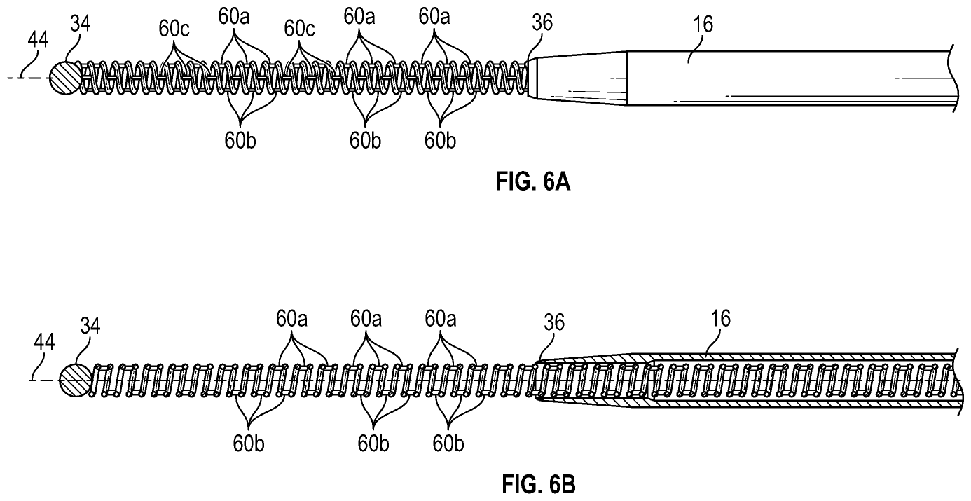

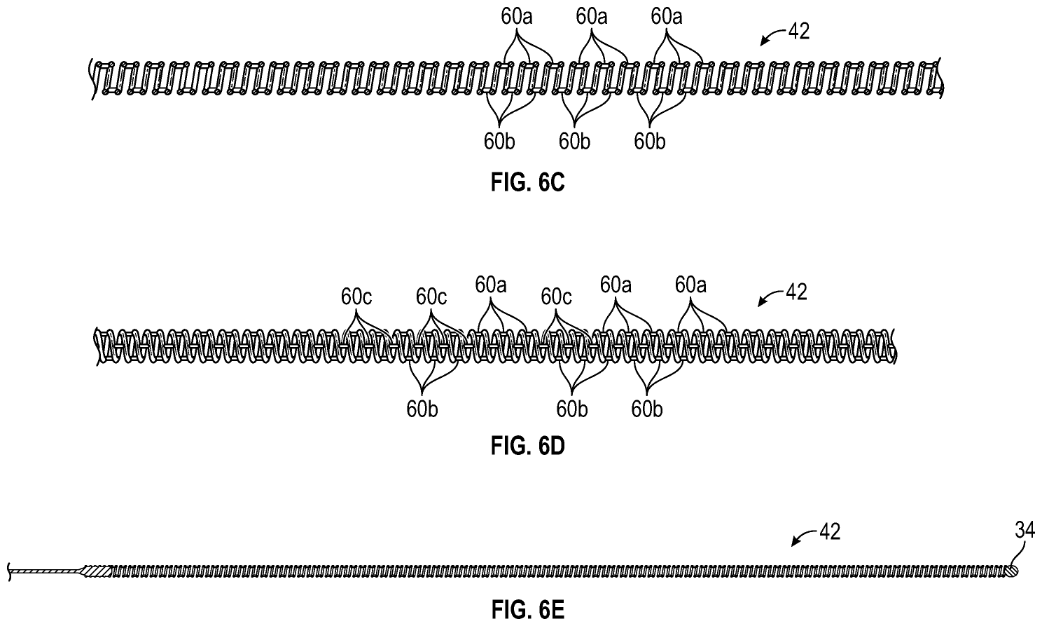

Referring now to FIGS. 6A-6E, in some embodiments, the vascular access instrument 32 may include multiple support beams 60 extending between the coil 42. In some embodiments, a first set of the support beams 60a may be disposed along a first line parallel to the axis 44. In some embodiments, a second set of the support beams 60b may be disposed along a second line parallel to the axis 44. In some embodiments, the first line may be spaced apart from the second line. In some embodiments, the first line may be 180 degrees apart from the second line, as illustrated in FIGS. 6A-6E.

In some embodiments, a third set of the support beams 60c may be disposed along a third line parallel to the axis 44. In some embodiments, the first line, the second line, and the third line may be evenly spaced around a circumference of the coil 42. In some embodiments, a complete turn of the coil 42 is disposed in between next adjacent support beams of the first set of support beams 60a. In some embodiments, a complete turn of the coil 42 is disposed in between next adjacent support beams of the second set of support beams 60b. In some embodiments, a complete turn of the coil 42 is disposed in between next adjacent support beams of the third set of support beams 60c.

In some embodiments, at least a portion of the vascular access instrument 32 may not include the core wire 48, which may facilitate blood flow through the vascular access instrument 32 and into the catheter 16. In some embodiments, the support beams 60 may be welded to the coil 42.

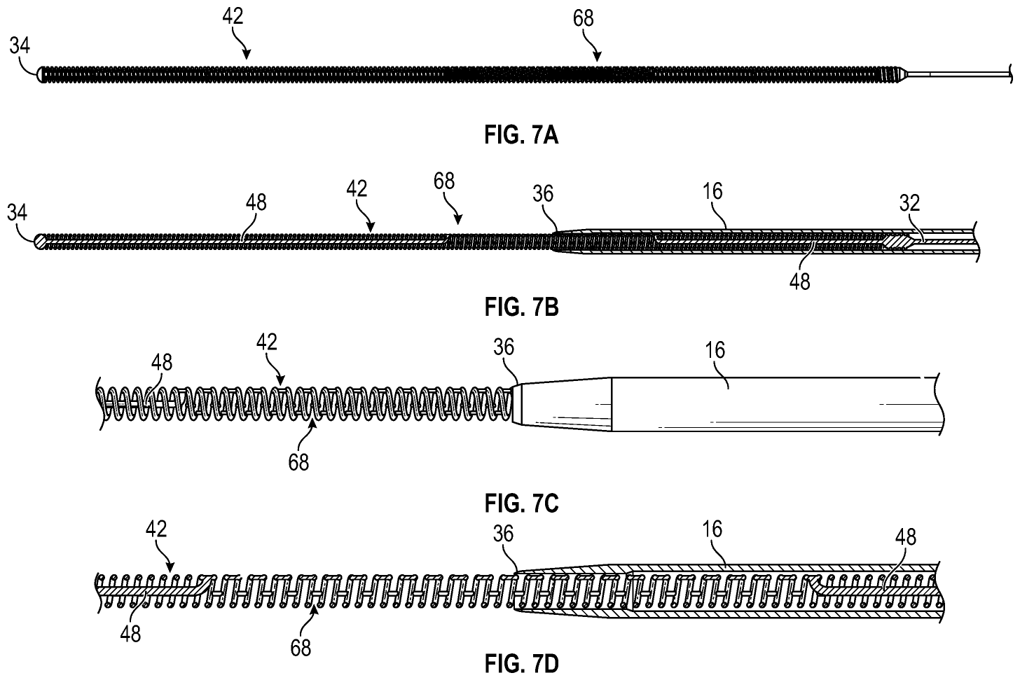

Referring now to FIGS. 7A-7D, in some embodiments, the core wire 48 may include one or more gaps 68 along a length of the coil 42. In some embodiments, a particular gap 68 in the core wire 48 may be disposed at the distal opening 53 of the catheter 16. In some embodiments, one or more ends of the core wire 48 may bend towards the coil 42 and/or be coupled to the coil 42. In some embodiments, the gaps 68 may correspond to locations where high shear stress on red blood cells and susceptibility to hemolysis is expected.

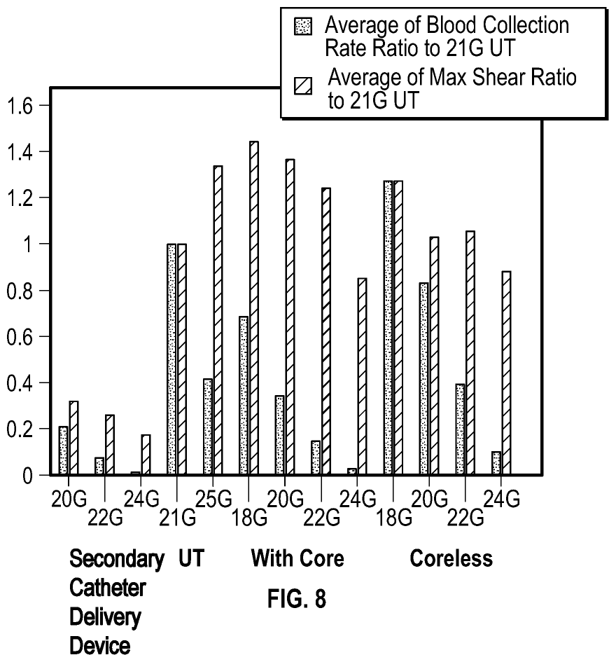

Referring now to FIG. 8, the bar graph illustrates, according to some embodiments, an average of blood collection rate ratio to 21G UT (“BD VACUTAINER™ ULTRATOUCH™”) and an average max shear ratio on blood cells to a 21G UT (“BD VACUTAINER™ ULTRATOUCH™”) in a particular vascular access system with the coil 42 that includes the flat wire wound around the axis 44 to form the loops 46, another particular vascular access system with the coil 42 that includes a cylindrical wire wound around the axis 44 to form the loops 46, and another particular vascular access system without the core wire 48.

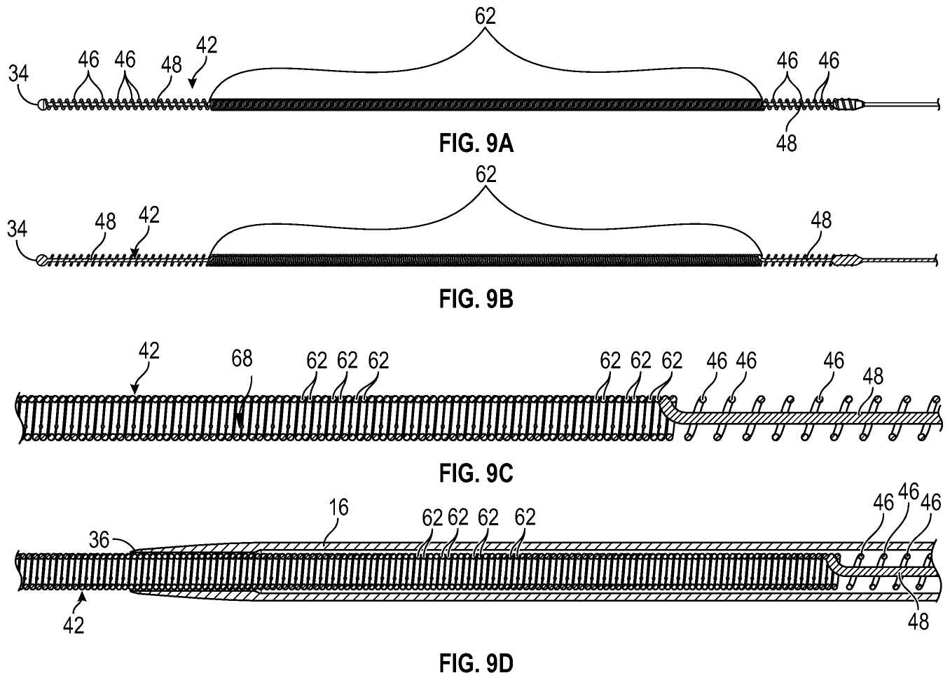

Referring now to FIGS. 9A-9D, in some embodiments, the coil 42 may include multiple other loops 62. In some embodiments, the other loops 62 may be proximate the loops 46. In some embodiments, each of the other loops 62 of the coil 42 may contact a next adjacent loop of the other loops 62 around a circumference of the next adjacent loop of the other loops 62. In some embodiments, a pitch of the coil 42 may change from the loops 46 with an open pitch to the other loops 62 with a closed pitch. In some embodiments, the loops 46 and the other loops 62 may be monolithically formed as a single unit. In some embodiments, the gap 68 in the core wire 48 may be disposed within the other loops 62. In some embodiments, the core wire 48 may begin again and/or be coupled to the coil 42 adjacent or proximate a proximal end of the other loops 62.

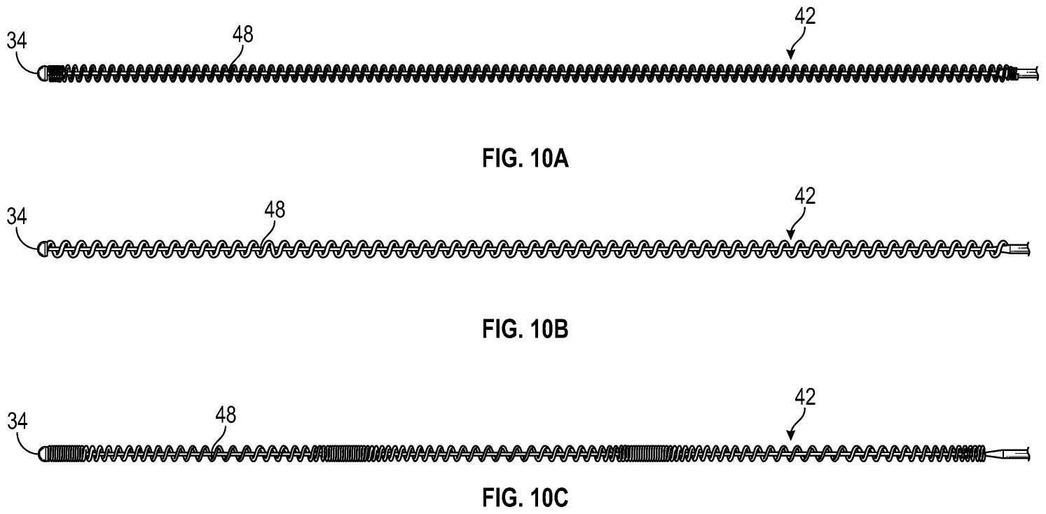

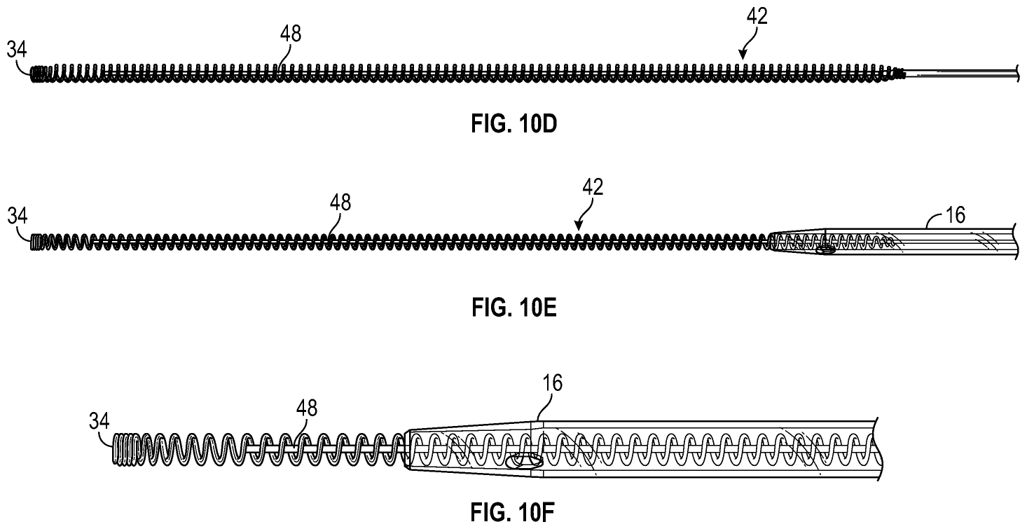

Referring now to FIGS. 10A-10F, in some embodiments, a pitch of the coil 42 may vary along a length of the coil 42. In some embodiments, the pitch may be less at ends of the coil 42, as illustrated, for example, in FIG. 10A. In some embodiments, the pitch may be less at the ends and/or one or more other portions of the coil 42 between the ends. In some embodiments, the other portions of the coil 42 may be evenly spaced, as illustrated, for example, in FIG. 10C. In some embodiments, the pitch of the coil 42 may be constant along the length of the coil, as illustrated, for example, in FIG. 10B. In some embodiments, the pitch may be greater or the loops 46 of the coil 42 may be more spaced apart in areas of high shear stress.

In some embodiments, a distal end of the core wire 48 may be spaced apart from the distal tip 34 of the vascular access instrument 32, which may facilitate fluid flow through the distal end of the vascular access instrument 32. In some embodiments, the distal tip 34 may be formed by the coil 42.

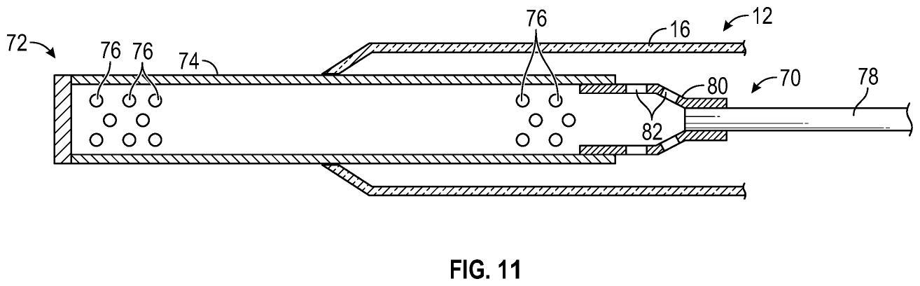

Referring now to FIG. 11, in some embodiments, another vascular access instrument 70 configured to insert through a vascular access device, such as the catheter assembly 12, may include a distal end 72, which may include a tubular element 74. In some embodiments, the tubular element 74 may include one or more holes 76. In some embodiments, the other vascular access instrument may include a wire 78. In some embodiments, the other vascular access instrument may include a connector 80 disposed between the tubular element 74 and the wire 78. In some embodiments, the connector 80 may include one or more other holes 82. In some embodiments, a distal end of the tubular element may be closed.

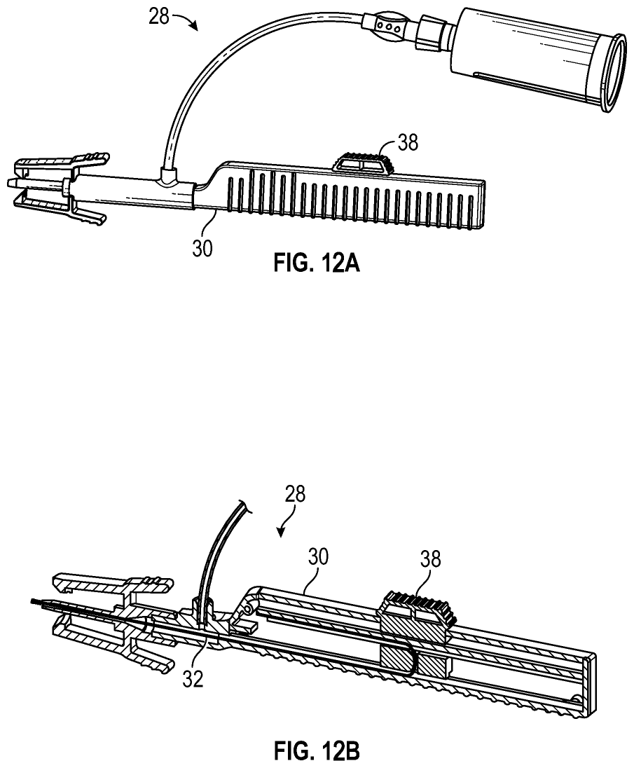

Referring now to FIGS. 12A-12B, the instrument advancement device 28 may include the housing 30 configured to couple to the catheter assembly 12. In some embodiments, the instrument advancement device 28 may include the vascular access instrument 32. In some embodiments, a proximal end of the vascular access instrument 32 may be coupled to an advancement tab 38, which may be gripped and moved along a slot 40 by a user to move the vascular access instrument 32 between the retracted position and the advanced position.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

We claim:

1. A vascular access instrument configured to insert through a vascular access device, the vascular access instrument comprising:
a coil formed by a flat wire wound around an axis into a plurality of loops, wherein each of the plurality of loops of the coil is spaced apart from a next adjacent loop of the plurality of loops.

2. The vascular access instrument of claim 1, further comprising a core wire extending through at least a portion of the coil.

3. The vascular access instrument of claim 2, wherein the core wire is cylindrical.

4. The vascular access instrument of claim 2, wherein the core wire is flat.

5. The vascular access instrument of claim 2, wherein the core wire is aligned with the axis.

6. The vascular access instrument of claim 2, wherein the core wire is offset from the axis.

7. The vascular access instrument of claim 6, wherein the flat wire comprises a first side and a second side opposite the first side, wherein the first side forms an outer surface of the coil, wherein the second side forms an inner surface of the coil, wherein the core wire is coupled to the inner surface of the coil.

8. The vascular access instrument of claim 1, further comprising an elongated strip extending through the coil, wherein each of the plurality of loops comprises a distal end and a proximal end, wherein the distal end and the proximal end contact the elongated strip.

9. The vascular access instrument of claim 1, further comprising a plurality of support beams extending between the coil, wherein a first set of the plurality of support beams is disposed along a first line parallel to the axis, wherein a second set of the plurality of support beams is disposed along a second line parallel to the axis, wherein the first line is spaced apart from the second line.

10. The vascular access instrument of claim 9, wherein the first line is 180 degrees apart from the second line.

11. The vascular access device of claim 9, wherein a third set of the plurality of support beams is disposed along a third line parallel to the axis, wherein the first line, the second line, and the third line are evenly spaced around a circumference of the coil.

12. The vascular access device of claim 11, wherein a complete turn of the coil is disposed in between next adjacent support beams of the first set, next adjacent support beams of the second set, and next adjacent support beams of the third set.

13. The vascular access device of claim 1, wherein the coil comprises another plurality of loops, wherein the other plurality of loops is proximate the plurality of loops, wherein each of the other plurality of loops of the coil contacts a next adjacent loop of the other plurality of loops along a circumference of the next adjacent loop of the other plurality of loops.

14. A vascular access system, comprising:
a catheter assembly, comprising a catheter adapter and a catheter extending distally from the catheter adapter;
an instrument advancement device coupled to the catheter assembly, wherein the instrument advancement device comprises a vascular access instrument, wherein the instrument advancement device is configured to advance the vascular access instrument from a retracted position to an advanced position beyond a distal end of the catheter, wherein the vascular access instrument comprises a coil formed by a flat wire wound around an axis into a plurality of loops, wherein each of the plurality of loops of the coil is spaced apart from a next adjacent loop of the plurality of loops, wherein the distal end of the catheter comprises a distal opening, wherein the coil extends through the distal opening of the catheter in response to the vascular access instrument being in the advanced position.

15. The vascular access system of claim 14, wherein the distal end of the catheter comprises a plurality of diffuser holes.

16. The vascular access system of claim 14, further comprising a core wire extending through the plurality of coils, further comprising a gap in the core wire at the distal opening of the catheter.

17. The vascular access system of claim 14, further comprising a plurality of support beams extending between the coil, wherein a first set of the plurality of support beams is disposed along a first line parallel to the axis, wherein a second set of the plurality of support beams is disposed along a second line parallel to the axis, wherein the first line is spaced apart from the second line.

18. The vascular access system of claim 14, wherein a complete turn of the coil is disposed in between next adjacent support beams of the first set and next adjacent support beams of the second set.

19. A vascular access instrument configured to insert through a vascular access device, the vascular access instrument comprising:
a distal end, comprising a tubular element, wherein the tubular element comprises a plurality of holes;
a wire; and
a connector disposed between the tubular element and the wire, wherein the connector comprises another plurality of holes.

20. The vascular access instrument of claim 19, wherein a distal end of the tubular element is closed.
